(19) United States
(12) Patent Application Publication
(10) Pub. No.: US 2026/0306458 A1
(43) Pub. Date: Oct. 8, 2026

(54) Pharmaceutical Combination of Oral Ivermectin and Mebendazole for Treatment of Refractory or Metastatic Pancreatic Cancers

(71) Applicant: Mike Zhou, Miami Beach, FL (US)

(72) Inventor: Mike Zhou, Miami Beach, FL (US)

(21) Appl. No.: 19/289,280

(22) Filed: Aug. 4, 2025

Related U.S. Application Data
(63) Continuation of application No. 19/171,260, filed on Apr. 5, 2025.

(51) Int. Cl. A61K 31/4184; A61K 31/7048

(52) U.S. Cl. CPC A61K 31/4184; A61K 31/7048

(57) ABSTRACT

This invention introduces a fixed-dose oral pharmaceutical combination of ivermectin and fenbendazole, and/or mebendazole, designed as a last-line therapy for patients with advanced, metastatic, or treatment-resistant disease. It is particularly intended for use in terminal-stage cancers—including solid tumors and hematologic malignancies—as well as immune-mediated inflammatory conditions and rare disorders. This standardized formulation, available in tablet, capsule, or oral liquid form, ensures consistent bioavailability. By repurposing well-established anthelmintic agents with documented safety profiles, this therapy engages broad-spectrum, multi-mechanistic actions to overcome tumor progression, drug resistance, chronic inflammation, and immune dysregulation across diverse disease categories, and is supported by 84 peer-reviewed mechanistic and early-clinical/preclinical studies, including 18 published in Nature-family journals, and 239 real-world clinical observations. These three repurposed agents collectively act through more than seventy-eight validated mechanisms, operating individually and in overlapping synergistic pathways, thereby establishing a robust scientific and clinical foundation for its use.

### 1. TECHNICAL FIELD

This invention pertains to the field of drug repurposing, focusing on fixed-dose pharmaceutical combinations of ivermectin and fenbendazole, and/or mebendazole. It establishes a standardized therapeutic platform with validated bioavailability and multi-mechanistic activity, offering a novel approach to treating resistant cancers, immune-related diseases, and rare conditions by leveraging complementary pharmacological effects. Although these agents have been studied individually for their broad-spectrum antiparasitic effects and emerging anticancer potential, no existing patent or regulatory framework has established a standardized fixed-dose combination therapy with defined bioavailability and validated multi-mechanistic activity.

### 2. SUMMARY OF THE INVENTION

The present invention introduces a fixed-dose oral pharmaceutical combination integrating ivermectin and fenbendazole, and/or mebendazole, intended for patients with advanced, metastatic, or refractory disease who have exhausted all available treatment options. This structured formulation leverages multi-mechanistic activity to offer a viable alternative where standard therapies fail.

While certain therapies incorporate multi-targeted strategies, this invention offers a unique fixed-dose oral regimen that leverages complementary pharmacological mechanisms to address tumor progression, drug resistance, chronic inflammation, and immune dysregulation.

This patent establishes a novel combination framework, integrating these compounds into a structured regimen, providing physicians with an additional therapeutic tool for managing treatment-resistant cancers, immune-mediated conditions, and rare diseases.

### 3. INDICATIONS

The present invention applies to a broad range of advanced, metastatic, or treatment-resistant diseases, designed for patients who have exhausted standard treatment options and require a last-line therapy leveraging multi-mechanistic synergy. The pharmaceutical composition, comprising ivermectin and fenbendazole, and/or mebendazole, is indicated for the following conditions: advanced or metastatic solid tumors, hematologic malignancies, immune-mediated inflammatory diseases, and rare conditions.

Advanced or metastatic solid tumors include, but are not limited to, respiratory system cancers such as various types of lung cancer; digestive system cancers such as cancers of the liver, stomach, intestine, pancreas, and bile ducts, as well as gastrointestinal stromal tumors (GIST); genitourinary system cancers such as cancers of the prostate, kidney, and bladder; gynecologic cancers such as cancers of the ovary, cervix, and breast; and neurologic tumors such as glioblastoma, astrocytoma, and oligodendroglioma.

Hematologic malignancies include, but are not limited to, acute myeloid leukemia (AML), chronic lymphocytic leukemia (CLL), lymphoma, multiple myeloma, polycythemia vera, and myelodysplastic syndromes (MDS).

Other cancers, immune-mediated inflammatory diseases, and rare conditions include, but are not limited to, neuroendocrine tumors, head and neck cancers, squamous cell carcinoma, melanoma, sarcomas, Wilms tumor, salivary gland carcinoma, spindle cell sarcoma, as well as immune-mediated and inflammatory conditions such as psoriasis and osteoarthritis, and rare conditions including macular degeneration and Parkinson's disease.

### 4. BACKGROUND TECHNOLOGY

Advanced cancer patients often lack therapeutic options after exhausting standard treatments including chemotherapy, radiotherapy, targeted therapies, and immunotherapies. In such cases, the progression to multi-organ metastases and disease deterioration leaves millions of patients worldwide with limited alternatives, contributing to high mortality rates. The absence of effective, affordable treatments represents a pressing medical challenge, while traditional drug development remains slow, costly, and unable to meet urgent clinical needs.

Over three decades of human and veterinary use have established the exceptional safety profiles of ivermectin, fenbendazole, mebendazole, providing a strong pharmacovigilance foundation for their adaptation into oncology. Ivermectin, approved for human use in 1987, has been administered to over 3.7 billion individuals globally. Fenbendazole, originally developed for veterinary applications, shares structural and mechanistic similarities with Mebendazole, an FDA-approved human antiparasitic drug since 1974. The extensive safety data supporting these compounds enables their clinical transition from compassionate off-label use into a formalized, regulatory-approved treatment pathway.

### 5. MODES OF IMPLEMENTATION

Under the supervision of a qualified physician, patients may follow the therapeutic protocol outlined in this invention by orally administering ivermectin and fenbendazole, and/or mebendazole either as separate formulations or within a fixed-dose combination capsule. To ensure accessibility and clinical adaptability, this invention prioritizes the use of readily available pharmaceutical-grade formulations, specifically:

FDA-approved human-use ivermectin, recognized for its established safety profile.

Veterinary-use Fenbendazole, repurposed under clinical discretion, with the option to substitute or co-administer Mebendazole, an FDA-approved human antiparasitic since 1974.

This invention establishes a scalable, accessible oncological treatment strategy, leveraging existing pharmaceutical compounds. By mitigating the challenges associated with slow and costly new drug development, this approach has the potential to address critical unmet medical needs among advanced cancer patients

### 6. MECHANISMS OF ACTION

The proposed therapeutic strategy utilizes a fixed-dose oral combination of ivermectin and fenbendazole, and/or mebendazole, which individually and collectively exert multi-targeted antitumor and anti-inflammatory activities. These agents inhibit tumor growth, overcome resistance mechanisms, and modulate the tumor microenvironment through a broad spectrum of molecular pathways.

The therapeutic effects arise from multiple coordinated actions demonstrated in extensive preclinical research. These include regulation of cell cycle and cell death through induction of G2/M phase arrest, activation of p53-mediated apoptosis, initiation of pyroptosis, and modulation of autophagy leading to cancer cell elimination. Mitochondrial and metabolic disruption is achieved through interference with glycolysis, glutaminolysis, and oxidative phosphorylation, resulting in cellular energy depletion and oxidative stress further enhanced by their combined action. Destabilization of microtubules and inhibition of DNA repair occur via disruption of mitotic spindle assembly and suppression of DNA damage repair pathways, leading to enhanced cancer cell sensitivity to apoptosis.

Immune and inflammatory modulation is also a critical feature, with enhancement of antitumor T-cell infiltration, suppression of immunosuppressive myeloid-derived suppressor cells, reduction of pro-inflammatory cytokines such as TNF-α and TGF-β1, and induction of immunogenic cell death, thereby improving immune surveillance. Oncogenic signaling pathways including Wnt/β-catenin, PI3K/Akt/mTOR, STAT3, MAPK/ERK, and NF-κB are suppressed, reducing proliferation, survival, and metastatic potential. Anti-angiogenic effects are observed through inhibition of hypoxia-inducible factors and restriction of tumor-associated vascularization, accompanied by remodeling of the tumor niche toward an immune-active and less permissive state.

These mechanisms extend beyond direct cytotoxic effects to include anti-inflammatory and immune-regulatory actions, supporting the applicability of this therapeutic approach in both malignant and inflammation-driven disorders. The described mechanisms have been validated across diverse tumor models and inflammatory disease settings, confirming their broad biological relevance.

Furthermore, preclinical studies evaluating the combined use of ivermectin and fenbendazole and/mebendazole have demonstrated synergistic effects, including enhanced mitochondrial disruption, inhibition of energy metabolism, induction of autophagy, and suppression of cancer stem cells. These synergistic interactions strengthen the rationale for employing this fixed-dose combination to achieve robust antitumor responses and improved disease control. Beyond the core mechanistic pathways described, additional biological processes have been identified that contribute to the overall therapeutic effects. These mechanisms collectively encompass more than seventy-eight validated anticancer, anti-inflammatory, and immune-regulatory pathways, as demonstrated by extensive peer-reviewed research across a wide range of experimental models. The findings summarized herein are supported by a large body of scientific literature, including numerous high-impact publications, and represent a robust foundation for the proposed therapeutic approach.

### Conclusion

The present invention proposes a fixed combination oral drug regimen consisting of ivermectin and fenbendazole, and/or mebendazole, designed for the last-line treatment of advanced, multi-organ metastatic, drug-resistant cancers, as well as immune-related inflammatory diseases and rare diseases. This regimen provides a structured therapeutic path for patients who have exhausted standard options, including those treated under compassionate-use conditions. It targets tumor proliferation, drug resistance, immune dysregulation, and chronic inflammation through multiple complementary mechanisms, ensuring broad-spectrum therapeutic applicability.

This invention is grounded in strong scientific rationale and real-world observational support, including 239 documented real-world observation case reports across more than thirty (30) cancer types and seven (7) non-oncology indications, and 84 peer-reviewed mechanistic and early-clinical/preclinical investigations validating its multi-pathway approach. While this approach has been applied in certain cases, including repurposed, off-label, or compassionate-use scenarios by practicing physicians, it has not yet been formalized as a standard treatment or patented. The present invention unifies these findings into a cohesive therapeutic platform, backed by multiple mechanistic studies, safety data, and clinical observations, thereby establishing a sound basis for intellectual property protection and further clinical development.

This combination regimen does not involve specific dosing recommendations but is founded on its scientifically validated mechanisms and broad-spectrum applicability, providing physicians with an additional therapeutic tool and offering new treatment possibilities for patients with severe disease progression. This therapy is safe, cost-effective, and clinically viable, establishing a scientifically robust and standardized treatment framework with comprehensive patent protection. This invention therefore defines a novel therapeutic paradigm that integrates repurposed agents into a standardized, multi-pathway oral regimen for patients lacking effective alternatives.

### REFERENCES

The invention is supported by extensive preclinical research, safety studies, regulatory data, economic analyses, and real-world clinical observations, detailed in a consolidated non-patent literature document submitted with this application.

1: A pharmaceutical composition comprising ivermectin and fenbendazole, and/or mebendazole, designed for the treatment of advanced, metastatic, or treatment-resistant diseases. This composition applies to solid tumors, hematologic malignancies, immune-mediated conditions, rare diseases, and other diseases or conditions, serving as a last-line therapy for patients who have exhausted standard medical options.

2: A pharmaceutical composition for oral administration comprising ivermectin and fenbendazole, and/or mebendazole, formulated as a composite oral tablet, composite oral capsule, or oral liquid suspension, with fixed or flexible dosing designed to optimize therapeutic synergy in treating advanced cancers, immune-mediated diseases, rare conditions, and other diseases or conditions.

3: A pharmaceutical composition comprising ivermectin and fenbendazole, and/or mebendazole, for the treatment of advanced or metastatic solid tumors, hematologic malignancies, immune-mediated inflammatory diseases, and rare conditions, including but not limited to respiratory system cancers such as various types of lung cancer; digestive system cancers such as cancers of the liver, stomach, intestine, pancreas, and bile ducts, as well as gastrointestinal stromal tumors (GIST); genitourinary system cancers such as cancers of the prostate, kidney, and bladder; gynecologic cancers such as cancers of the ovary, cervix, and breast; neurologic tumors such as glioblastoma, astrocytoma, and oligodendroglioma; hematologic malignancies such as acute myeloid leukemia, chronic lymphocytic leukemia, lymphoma, multiple myeloma, polycythemia vera, and myelodysplastic syndromes; and other cancers and rare conditions including but not limited to neuroendocrine tumors, head and neck cancers, squamous cell carcinoma, melanoma, sarcomas, Wilms tumor, salivary gland carcinoma, spindle cell sarcoma, psoriasis, osteoarthritis, macular degeneration, and Parkinson's disease.
